(19) United States
(12) Patent Application Publication
(10) Pub. No.: US 2003/0054025 A1
(43) Pub. Date: Mar. 20, 2003

(54) Non-contact printing method for making a medical pressure sensitive adhesive article

(75) Inventors: Adam S. Cantor, St. Paul, MN (US); Albert I. Everaerts, Oakdale, MN (US); Jeffrey H. Tokie, Scandia, MN (US)

Correspondence Address: Attention: Ted K. Ringsred, Office of Intellectual Property Counsel, 3M Innovative Properties Company, P.O. Box 33427, St. Paul, MN, 55133-3427, US

(73) Assignee: 3M Innovative Properties Company

(21) Appl. No.: 10/242,065

(22) Filed: Sep. 12, 2002

Related U.S. Application Data
(60) Provisional application No. 60/322,306, filed on Sep. 14, 2001.

(51) Int. Cl. A61L 15/16; A61K 9/70; B32B 15/04

(52) U.S. Cl. 424/448; 428/343; 156/250; 424/449

(57) ABSTRACT

This invention relates to non-contact printing methods of making medical pressure sensitive adhesive articles. A base layer is coated onto a substrate and a liquid composition is non-contact printed onto at least a portion of the base layer to provide a medical pressure sensitive adhesive article. The medical pressure sensitive adhesive article comprises at least one medicinal ingredient intended to have a medicinal or therapeutic effect. More specifically this invention relates to non-contact printing methods of making transdermal drug delivery devices.

This application claims the benefit of Serial No. 60/322,306, filed Sep. 14, 2001.

### FIELD OF THE INVENTION

This invention relates to a non-contact printing method for making medical pressure sensitive adhesive articles. More specifically this invention relates to a non-contact printing method for making transdermal drug delivery devices.

### BACKGROUND OF THE INVENTION

Pressure sensitive adhesive articles typically consist of thin layers of pressure sensitive adhesive (PSA) material that have been applied or prepared on a substrate. When the substrate is a flexible backing material, the coated article is a pressure sensitive adhesive sheet, such as a tape or label.

Medical PSA sheets are well known and include bandages, first-aid dressings, wound care dressings, transdermal drug delivery devices and the like. There are often special requirements for medical PSA sheets in addition to the fundamental necessity that they adhere to skin or other body tissues. They often contain ingredients in addition to the polymeric matrix that provides the basis for PSA properties. These additives include antimicrobials, antifungals, drugs and the like that are intended to have a medicinal or therapeutic effect.

One particular type of medical sheet containing additives is a transdermal drug delivery device. A variety of transdermal drug delivery devices have been described. Devices known to the art include reservoir type devices involving membranes that control the rate of drug and/or skin penetration enhancer delivery to the skin, single layer devices involving a dispersion or solution of drug and excipients in a pressure sensitive adhesive matrix, and more complex multilaminate devices involving several distinct layers, e.g., layers for containing drug, for containing skin penetration enhancer, for controlling the rate of release of the drug and/or skin penetration enhancer, and for attaching the device to the skin.

When a transdermal drug delivery device incorporates a pressure sensitive adhesive layer comprising a dispersion or solution of drug and/or an excipient in a polymeric matrix, the pressure sensitive adhesive layer typically is prepared by solution coating. That is, dissolving the polymer and the drug and/or excipient in a solvent, coating the resulting solution onto a web, and then oven drying the coated web to evaporate the solvent. This conventional method has several disadvantages. For example, if the drug and/or excipient is volatile or heat sensitive, then the oven drying step can lead to loss of drug and/or excipient due to either evaporation or to thermal degradation. Also, this method typically involves using a volatile organic solvent which may be undesirable for environmental and health reasons.

### SUMMARY OF THE INVENTION

The present invention provides a method of preparing a medical pressure sensitive adhesive article. In the first step of the method, a base layer on a substrate is provided. The base layer comprises a pressure sensitive polymer adhesive. In the second step, a liquid composition comprising at least one medicinal ingredient intended to have a medicinal or therapeutic effect is non-contact printed onto at least a portion of the base layer, and at least a portion of the liquid composition is allowed to diffuse into the base layer to provide a medical pressure sensitive adhesive article. In a preferred embodiment of the present invention the medical pressure sensitive adhesive article is a medical pressure sensitive adhesive sheet.

In another preferred embodiment of the present invention the medicinal ingredient intended to have a medicinal or therapeutic effect is a drug.

The invention further provides transdermal drug delivery devices prepared by the methods described herein.

In another aspect, the invention provides a method of preparing a medical pressure sensitive adhesive article wherein, in one step, a base layer on a substrate is provided. The base layer comprises a polymer. In a separate step, a liquid composition comprising at least one medicinal ingredient intended to have a medicinal or therapeutic effect is non-contact printed onto at least a portion of a flexible film. Subsequently, the base layer is laminated to the printed liquid composition and at least a portion of the liquid composition is allowed to diffuse into the base layer to provide a medical pressure sensitive adhesive article.

In yet another aspect, the invention provides a method of preparing a medical pressure sensitive adhesive article wherein, in the first step, a base layer comprising a polymer and at least one medicinal ingredient intended to have a medicinal or therapeutic effect is provided on a substrate. In the second step, a liquid composition is non-contact printed onto at least a portion of the base layer, and at least a portion of the liquid composition is allowed to diffuse into the base layer to provide a medical pressure sensitive adhesive article. In a preferred embodiment the base layer prepared in the first step is non-tacky.

In still another aspect, the invention provides a method of preparing a medical pressure sensitive adhesive article wherein a base layer comprising a polymer and at least one medicinal ingredient intended to have a medicinal or therapeutic effect is provided on a substrate in one step. In a separate step, a liquid composition is non-contact printed onto at least a portion of a flexible film. Subsequently, the base layer is laminated to the printed liquid composition and at least a portion of the liquid composition is allowed to diffuse into the base layer to provide a medical pressure sensitive adhesive article. In a preferred embodiment the base layer prepared in the first step is non-tacky.

This invention provides a number of advantages over previously known methods for preparing medical pressure sensitive adhesive articles, and more particularly for preparing transdermal drug delivery devices. These advantages include, but are not limited to, the ability to avoid exposure of medicinally active ingredients to excessive heat; the ability to prepare medical articles with varying spatial concentrations of the formulation components; the ability to prepare base layers using processing methods that are incompatible with the medicinally active ingredients; the ability to apply very precise and metered amounts of medicinally active ingredients; the ability to segregate potentially incompatible ingredients; the ability to “activate” a medical article from non-PSA to PSA just prior to dispensing or using; and providing a beneficial flexibility for manufacturing operations allowing for reduced cost and improved efficiency.

### DETAILED DESCRIPTION OF THE INVENTION

The present invention provides a method of preparing a medical pressure sensitive adhesive article.

In the first step of the method, a base layer on a substrate is provided. The base layer comprises a polymer, preferably a polymer selected from the group consisting of acrylates, natural rubbers, synthetic rubbers such as polyisobutylenes, polyisoprenes, styrenic block copolymers, polyvinylethers, and silicone polymers. The base layer polymers can be present alone or in combination. The base layer may optionally contain other additives, for example, medicinal ingredients, tackifiers, plasticizers, anti-oxidants, colorants, and the like.

Acrylate polymers and copolymers are preferred base layer polymers for use in the articles of the invention. Suitable acrylate copolymers for use in the base layer preferably comprise one or more A monomers selected from the group consisting of alkyl (meth)acrylates and aromatic or cycloalkyl (meth)acrylates. Useful alkyl acrylates include monofunctional unsaturated monomers selected from the group consisting of acrylate esters of non-tertiary alkyl alcohols, the alkyl groups containing 1 to 18 carbon atoms in the alkyl group, preferably about 4 to about 12 carbon atoms and mixtures thereof. Examples of suitable alkyl acrylates include methyl, ethyl, n-butyl, n-pentyl, n-hexyl, isoheptyl, n-nonyl, n-decyl, isohexyl, isodecyl, isooctyl and 2-ethylhexyl acrylates. Preferred alkyl acrylates include isooctyl acrylate, 2-ethylhexyl acrylate, and n-butyl acrylate. Useful alkyl methacrylates include monofunctional unsaturated monomers selected from the group consisting of methacrylate esters of non-tertiary alkyl alcohols, the alkyl groups containing 1 to 18 carbon atoms in the alkyl group, preferably 1 to about 12 carbon atoms and mixtures thereof. Examples of suitable alkyl methacrylates include methyl methacrylate, ethyl methacrylate, n-butyl methacrylate, n-hexyl methacrylate, noctyl methacrylate, isooctyl methacrylate and decyl methacrylate. Examples of suitable aromatic monomers and normal or bridged cycloalkyl (meth)acrylates include isobomyl acrylate, isobomyl methacrylate, 4-t-butylcyclohexyl methacrylate, cyclohexyl acrylate, cyclohexyl methacrylate, phenyl acrylate, 2-naphthyl acrylate, and 2-naphthyl methacrylate. Acrylate copolymers preferably comprise about 45 to about 95 percent by weight, more preferably 55 to 95 percent by weight, based on the total weight of all monomers in the copolymer, of one or more A monomers.

Acrylate copolymers may optionally further comprise one or more B monomers. Suitable B monomers include those containing a functional group selected from the group consisting of carboxylic acid, sulfonamide, urea, carbamate, carboxamide, hydroxy, amino, oxy, and cyano. Exemplary B monomers include acrylic acid, methacrylic acid, maleic acid, a hydroxyalkyl acrylate containing 2 to 4 carbon atoms in the hydroxyalkyl group, a hydroxyalkyl methacrylate containing 2 to 4 carbon atoms in the hydroxyalkyl group, acrylamide, methacrylamide, an alkyl substituted acrylamide containing 1 to 8 carbon atoms in the alkyl group, N-vinyl formamide, N-vinyl acetamide, N-vinyl valerolactam, N-vinyl caprolactam, N-vinyl-2-pyrrolidone, glycidyl methacrylate, vinyl acetate, alkoxyethyl acrylate containing 1 to 4 carbon atoms in the alkoxy group, alkoxyethyl methacrylate containing 1 to 4 carbon atoms in the alkoxy group, 2-ethoxyethoxyethyl acrylate, furfuryl acrylate, furfuryl methacrylate, tetrahydrofurfuryl acrylate, tetrahydrofurfuryl methacrylate, propylene glycol monomethacrylate, propylene oxide methyl ether acrylate, acrylonitrile, methacrylonitrile, di(lower alkyl)amino ethyl acrylate, di(lower alkyl)amino ethyl methacrylate, and di(lower alkyl)aminopropyl methacrylamide, where (lower alkyl) in the preceding examples represents alkyl groups containing 1 to 4 carbon atoms. Preferred B monomers include acrylic acid, methacrylic acid, acrylamide, 2-hydroxy ethyl acrylate, and vinyl acetate. Acrylate copolymers preferably comprise about 5 to about 55 percent by weight, more preferably about 5 to about 45 percent by weight, based on the total weight of all monomers in the copolymer, of one or more B monomers.

Acrylate copolymers may optionally further comprise a substantially linear macromonomer copolymerizable with the A and B monomers and having a weight average molecular weight in the range of about 500 to about 100,000, preferably about 2,000 to about 50,000 and more preferably about 5,000 to about 30,000. The macromonomer, when used, is generally present in an amount of not more than about 20%, preferably not more than about 10% by weight based on the total weight of all monomers in the copolymer. Suitable macromonomers include polymethylmethacrylate, styrene/acrylonitrile copolymer, polyether, and polystyrene macromonomers. Examples of useful macromonomers and their preparation are described in Krampe et al., U.S. Pat. No. 4,693,776, the disclosure of which is incorporated herein by reference.

The base layers of the present invention may have adhesive properties ranging from pressure sensitive to non-tacky depending on the types and amounts of polymers used, types and amounts of any other additives, and any other processing treatment, such as curing, applied to the base layer.

In a preferred embodiment of the invention the polymer of the base layer is a pressure sensitive adhesive, preferably an acrylate copolymer pressure sensitive adhesive, more preferably having an inherent viscosity in the range of about 0.2 dl/g to about 2 dl/g, more preferably about 0.5 dl/g to about 1.6 dl/g.

The copolymers described above can be prepared by methods well known to those skilled in the art and described for example in U.S. Pat. No. RE 24,906 (Ulrich), U.S. Pat. No. 4,554,324 (Husman et al.), U.S. Pat. No. 4,732,808 (Krampe et. al.), and International Publication Number WO 96/08229 (Garbe et. al.), the disclosures of which are incorporated herein by reference.

Another copolymerization method is photopolymerization of the monomer mixture initiated by ultraviolet (UV) radiation. This monomer mixture, along with a suitable photoinitiator, is coated onto a flexible carrier web and polymerized in an inert (i.e., oxygen free) atmosphere. A sufficiently inert atmosphere can be achieved by covering a layer of the photoactive coating with a plastic film which is substantially transparent to UV radiation, and irradiating through that film using fluorescent-type UV lamps.

Other solventless polymerization methods, such as the continuous free radical polymerization method described in U.S. Pat. No. 4,619,979 (Kotnour et. al.) and U.S. Pat. No. 4,843,134 (Kotnour et. al.); the essentially adiabatic polymerization methods using a batch reactor described in U.S. Pat. No. 5,637,646 (Ellis); and, the methods described for polymerizing packaged polymerizable mixtures described in U.S. Pat. No. 5,804,610 (Hamer et. al.) may also be utilized to prepare the base layer copolymers of the present invention.

To enhance cohesive strength, the base layer may optionally be subjected to additional radiation curing treatment, such as ultraviolet or electron beam irradiation. Alternatively, chemical curing methods, such as those introducing covalent crosslinking, ionic crosslinking, or physical crosslinking may also be optionally used.

The base layer polymers can be applied to any suitable substrate that can be a sheet material or a shaped article. In a preferred embodiment the substrate is a flexible material and the medical PSA article is a medical PSA sheet. The base layer polymers of the present invention are easily coated upon suitable flexible or inflexible substrates by conventional coating techniques, such as roll coating, spray coating, curtain coating, and the like to produce coated polymeric sheet materials in accord with the present invention. The base layer polymers may also be coated without modification by extrusion coating, coextrusion, hot-melt coating and the like by employing suitable conventional coating devices for this purpose. The base layer polymers may also be prepared in place on the substrate, for example using the photopolymerization method described above. The base layer polymers may be optionally coated as discontinuous patterns using conventional printing methods, such as flexographic printing or screen printing.

Sheet materials can be provided as individual pieces or as a continuous web, for example in a rollstock form. Preferred substrates are made from any material conventionally utilized as a tape backing, release liner, or any other flexible material. Typical examples of flexible backing materials employed as conventional tape backings which may be useful for the present invention include those made of paper, plastic films such as polypropylene, polyethylene, particularly low density polyethylene, linear low density polyethylene, metallocene polyethylenes, high density polyethylene, polyvinyl chloride, polyester (e.g., polyethylene terephthalate), ethylene-vinyl acetate copolymer, polyurethane, cellulose acetate and ethyl cellulose. Backings that are layered such as polyethylene terephthalate-aluminum-polyethylene composites are also suitable.

Backings may also be prepared of fabric such as woven fabric formed of threads of synthetic or natural materials such as cotton, nylon, rayon, glass, ceramic material, and the like or nonwoven fabric such as air laid webs of natural or synthetic fibers or blends of these.

In a particularly preferred embodiment the flexible substrate is a release liner. Suitable release liners include conventional release liners comprising a known sheet material such as a polyester web, a polyethylene web, a polypropylene web, or a polyethylene-coated paper coated with a suitable fluoropolymer or silicone based coating.

In the second step, a liquid composition comprising at least one medicinal ingredient intended to have a medicinal or therapeutic effect is non-contact printed onto at least a portion of the base layer, and the liquid composition is allowed to diffuse into the base layer to provide a medical pressure sensitive adhesive article. This technique, where the liquid composition is non-contact printed directly onto the base layer will be referred to hereafter as the “direct printing” technique.

In contrast to contact printing methods, such as gravure, pin stamp, or rubber stamp, a non-contact printing method employs fluid dispensing mechanisms that may be moved in a transverse direction to the substrate while imparting substantially no transverse force to the substrate. In some embodiments, such as ink jet printing, the fluid dispensing mechanism and the substrate are not in direct fluid communication, being separated by one or more gaps of air, or another gas, such as nitrogen in the case where a nitrogen purged environment is used. In other embodiments, such as needle jet printing, a continuous stream of fluid emerging from the fluid dispensing mechanisms may simultaneously contact the dispensing mechanism and the substrate. In those embodiments, however, the continuous stream of fluid will have a combination of a sufficiently large aspect ratio (length divided by cross sectional area) and a sufficiently low transverse shear modulus, so that movement of the dispensing mechanism in a direction transverse to the substrate will transmit substantially no transverse force to the substrate. It should be understood that substantially no transverse force is defined as any transverse force that does not interfere with or degrade the registration between the fluid dispensing mechanism and the substrate.

The printer includes one or more fluid dispensing mechanisms and a substrate handling system designed to receive and present the base layer to the fluid dispensing mechanisms. The fluid dispensing mechanism(s), preferably an ink jet print head, is mounted so that it is adjustable with respect to the base layer.

The fluid dispensing mechanisms used for non-contact printing may include ink-jetting, valve-jetting, needle-jetting, spray-jetting, stream-jetting, ultrasonic atomization, and the like. Combinations of each of these fluid dispensing mechanisms may also be used to achieve desired dispensing rates and volumes. These methods also allow for precision printing of fluids in high resolution patterns.

Inkjet printing is a preferred non-contact printing method. Ink jet printers operate by ejecting a fluid onto a receiving substrate in controlled patterns of fluid droplets. Ink jet printing includes continuous and impulse jetting methods, including thermal, bubble, drop-on-demand and piezo ink jet.

Needle-jetting is another preferred non-contact printing method. Needle jetting operates by opening, closing or varying a valve in fluid communication with an orifice. The emitted fluid is usually observed in the form of a stream or steady flow of droplets which is directed onto a receiving substrate. The orifice, valve, backpressure and fluid properties control the volumetric flow. Alternative methods for controlling flow may also be used, such as piston/cylinder positive displacement systems, gear pumps or similar mechanical devices. These latter methods may require the use of a set orifice diameter which is variable only by changing the mechanical parts. (Same thing for Spray jetting).

Spray-jetting is still another preferred non-contact printing method. Spray-jetting uses the same flow regulating mechanisms as needle jetting, valve-jetting, and stream jetting, and includes the use of an air impingement nozzle or air stripping nozzle to create atomization to some degree. Spray jetting may also use vibration to create an atomized fluid stream. The emitted fluid is directed onto a receiving substrate.

In the present invention, the receiving substrate for the liquid composition is the base layer coated in step one of this method described above. The base layer may be brought into close proximity with the fluid dispensing mechanism by any number of conventional methods. One preferred method for bringing the base layer into close proximity with the fluid dispensing mechanism is by employing a substrate handling system having a rotatable drum driven by a drum motor for use in presenting and controlling the positioning of the base layer onto which the liquid composition is to be applied. The diameter and length of the drum may vary depending on the desired size of the substrate to be used and the overall desired size of the printer. The drum may optionally include a vacuum system or may be heated or cooled by conventional methods well known in the art. The receiving base layer may also be adjustably positioned, for example by adjustably moving the drum in relation to the fluid dispensing mechanism.

Another preferred method for bringing the base layer into close proximity with the fluid dispensing mechanism is by handling the substrate on a platen so that the printing device becomes part of a flat bed operation. The print pattern can be changed by a simple axial movement of the fluid dispensing mechanism or platen in relation to each other. The platen may have a vacuum hold-down or other pinning method for the substrate and it may be heated or cooled by methods well known in the art. Other methods to move the substrate relative to the fluid dispensing mechanism can also be used. They include mechanical mechanisms, such as driven rollers to move the substrate, and moving printheads, such as for example used in a desk top printer.

One or more control systems are provided to control the fluid dispensing mechanism(s) and the substrate handling system. The control systems preferably employ a computer, microprocessor, or other electronic control device. In a preferred embodiment a single overall control system connects to both the fluid dispensing mechanism(s) and the substrate handling system.

The viscosity of the liquid composition should be suitable for non-contact printing, and more preferably for ink jet printing as is conventionally known in the art. The liquid composition may be heated to adjust the viscosity for printing. Generally, the liquid composition should be of such a chemical nature as to allow it to diffuse into the base layer. After printing, the liquid composition is allowed to diffuse into the base layer until the prepared medical PSA article is sufficiently dry to allow for lamination, wind-up, or storage. The length of time that this takes is dependent on the type of base layer, the type of coating liquid, and the relative amounts of base layer and coating liquid. This time is preferably less than about 5 to about 10 minutes and more preferably less than about one minute. To facilitate and accelerate diffusion of the liquid composition, it may be advantageous to heat the base-layer just before, during, and/or after deposition of the liquid composition.

The liquid composition comprises at least one medicinal ingredient intended to have a medicinal or therapeutic effect. The medicinal ingredient may be a liquid, in which case the liquid composition may consist entirely of the medicinal ingredient if the viscosity of the medicinal ingredient is appropriate for non-contact printing. The medicinal ingredient may also be mixed with other solids or liquids to prepare the liquid composition by any conventional method known to those skilled in the art. In particular, a solid medicinal ingredient may be dissolved or dispersed in a liquid or mixture of liquids to prepare the liquid composition.

Preferably, no additional drying or curing steps are needed to prepare the medical PSA article after allowing the liquid composition to diffuse into the base layer. The liquid composition may, however, optionally include a volatile organic solvent as a carrier for other materials. In this instance the volatile organic solvent is subsequently removed from the base layer after the other materials that it carried are allowed to diffuse into the base layer. The combination of the base layer and the liquid composition may optionally be subjected to additional curing treatment, such as ultraviolet or electron beam irradiation to achieve desired PSA properties.

The liquid composition is sufficiently miscible with the materials of the base layer to allow at least a portion of it to diffuse into the base layer. Preferably all of the liquid composition will diffuse into the base layer. The liquid composition is preferably miscible with the materials of the base layer in the amounts used in the final composition of the medical PSA article. Examples of suitable liquid compositions can be softening agents (e.g., plasticizers), skin penetration enhancers, solubilizers, drugs or other medicinal ingredients.

The liquid composition may be coated uniformly across the base layer or it may be coated in any of a variety of high precision controlled patterns. In a preferred embodiment the liquid composition is printed in island patterns onto a continuous web. These island patterns may be sized such that discrete pressure sensitive tape patches may be converted from the continuous web in such fashion that the base layer web remaining after all of the discrete pressure sensitive tape patches are converted is substantially free of the liquid composition. The converting of the continuous web into discrete pressure sensitive tape articles may take place in a batch process whereby the island pattern coated continuous web is rolled up on itself or with a release liner to form rollstock. The rollstock is subsequently unwound to allow for cutting or punching of the discrete pressure sensitive tape articles from the continuous web. In a preferred embodiment, the converting of the continuous web into discrete pressure sensitive tape patches may take place in an integrated process where the discrete pressure sensitive tape patches are cut or punched from the web in a step prior to formation of rollstock.

The medical PSA article that is provided as a result of the second step comprises at least one medicinal ingredient intended to have a medicinal or therapeutic effect. This medicinal ingredient may be a drug, antimicrobial agent, antifungal agent, cosmetic agent, or pharmaceutically effective excipient.

In a preferred embodiment, the medicinal ingredient is a drug. In a particularly preferred embodiment the medical PSA article will comprise a transdermal drug delivery composition. The drug will be present in an amount such that the composition delivers a therapeutically effective amount for the condition being treated. This amount will vary according to the type of drug used, the condition to be treated, the amount of time the composition is allowed to remain in contact with the skin of the subject, and other factors known to those of skill in the art. However, the amount of drug present in the transdermal drug delivery composition of the invention will generally be about 0.01 to 40 wt-%, preferably about 1.0 to 20 wt-%, based on the total weight of the composition. In a composition of the invention the drug is dispersed, preferably homogeneously, and more preferably dissolved in the pressure sensitive adhesive.

Any drug that is suitable for transdermal delivery may be used in the transdermal drug delivery composition of the invention. Examples of useful drugs include, but are not limited to, antiinflammatory drugs, both steroidal (e.g., hydrocortisone, prednisolone, triamcinolone) and nonsteroidal (e.g., naproxen, piroxicam); antibacterials (e.g., penicillins such as penicillin V, cephalosporins such as cephalexin, erythromycin, tetracycline, gentamycin, sulfathiazole, nitrofurantoin, and quinolones such as norfloxacin, flumequine, and ibafloxacin); antiprotozoals (e.g., metronidazole); antifungals (e.g., nystatin); coronary vasodilators (e.g., nitroglycerin); calcium channel blockers (e.g., nifedipine, diltiazem); bronchodilators (e.g., theophylline, pirbuterol, salmeterol, isoproterenol); enzyme inhibitors such as collagenase inhibitors, protease inhibitors, elastase inhibitors, lipoxygenase inhibitors (e.g., zileuton), and angiotensin converting enzyme inhibitors (e.g., captopril, lisinopril); other antihypertensives (e.g., propranolol); leukotriene antagonists; antiulceratives such as H2 antagonists; steroidal hormones (e.g., progesterone, testosterone, estradiol); antivirals and/or immunomodulators (e.g., 1-isobutyl-1H-imidazo[4,5-c]quinolin-4-amine, 1-(2-hydroxy-2-methylpropyl)-1H-imidazo[4,5-c]quinoline-4-amine, and other compounds disclosed in U.S. Pat. No. 4,689,338, incorporated herein by reference, acyclovir); local anesthetics (e.g., benzocaine, propofol); cardiotonics (e.g., digitalis, digoxin); antitussives (e.g., codeine, dextromethorphan); antihistamines (e.g., diphenhydramine, chlorpheniramine, terfenadine); narcotic analgesics (e.g., morphine, fentanyl); peptide hormones (e.g., human or animal growth hormones, LHRH); sex hormones (e.g., estrogens, testosterone, progestins such as levonorgestrel, norethindrone, gestodene); cardioactive products such as atriopeptides; proteinaceous products (e.g., insulin); enzymes (e.g., antiplaque enzymes, lysozyme, dextranase); antinauseants (e.g., scopolomine); anticonvulsants (e.g., carbamazine); immunosuppressives (e.g., cyclosporine); psychotherapeutics (e.g., diazepam); sedatives (e.g., phenobarbital); anticoagulants (e.g., heparin); analgesics (e.g., acetaminophen); antimigraine agents (e.g., ergotamine, melatonin, sumatriptan); antiarrhythmic agents (e.g., flecainide); antiemetics (e.g., metaclopromide, ondansetron); anticancer agents (e.g., methotrexate); neurologic agents such as anxiolytic drugs; hemostatics; anti-obesity agents; and the like, as well as pharmaceutically acceptable salts and esters thereof.

Pressure sensitive tapes may be prepared by laminating a release liner to a medical PSA sheet to cover at least a portion of the PSA. Where the base layer was initially applied to a release liner, a pressure sensitive tape may be prepared by laminating a backing to the medical PSA sheet. Suitable flexible backing materials employed as conventional tape backings are described above.

In another preferred embodiment the medicinal ingredient may be an antimicrobial agent. Antimicrobial agents may include any broad spectrum antimicrobial agent that is suitable for topical application. Examples of suitable antimicrobial agents are iodine, including iodine complexes with sodium or potassium iodide as well as polymeric complexes often called iodophors such as povidone-iodine and polyethylene glycol complexes, hexylresorcinol, chlorhexidine or a suitable salt thereof such as chlorhexidine gluconate or chlorhexidine acetate, triclosan, p-chloro-m-xylenol (PCMX), phenols, peroxides, silver and silver salts such as silver chloride, silver oxide and silver sulfadiazine, long chain alkyl quaternary ammonium compounds, and mono C₈-C₁₂ alkyl esters of glycerin and propylene glycol. Antifungal agents may also be incorporated and include any of the “azoles” such as miconazole nitrate, chlortrimazole, econazole, ketoconizole and the like as well as tolnaftate and undecylic acid and its salts. Iodine, iodine complexes with sodium or potassium iodide, povidone-iodine, and chlorhexidine are preferred antimicrobial agents.

In another preferred embodiment the medicinal ingredient may be a cosmetic agent. Examples of cosmetic agents may include alpha hydroxy acids, beta hydroxy acids, glycolic acid, essential amino acids, essential fats, homeopathic agents, botanicals, coenzyme Q10, flavonoids, fragrance, anti-acne agents, antihyperpigmentation agents, soothing agents, sunburn agents, free radical scavengers, seboregulators, hydratives, vitamins, antioxidants, anti-irritants and minerals. Vitamins are preferred cosmetic agents.

In another preferred embodiment the medicinal ingredient may be a pharmaceutically or medically effective excipient. These excipients may include softening agents (softeners) used as skin penetration enhancers or solubilizers in transdermal drug delivery systems. Exemplary materials include C₈-C₃₆ fatty acids such as isostearic acid, octanoic acid, and oleic acid; C₈-C₃₆ fatty alcohols such as oleyl alcohol and lauryl alcohol; lower alkyl esters of C₈-C₃₆ fatty acids such as ethyl oleate, isopropyl myristate, butyl stearate, and methyl laurate; di(lower) alkyl esters of C₆-C₈ diacids such as diisopropyl adipate; monoglycerides of C₈-C₃₆ fatty acids such as glyceryl monolaurate; tetraglycol (tetrahydrofurfuryl alcohol polyethylene glycol ether); tetraethylene glycol (ethanol,2,2′-(oxybis(ethylenoxy))diglycol); C₆-C₃₆ alkyl pyrrolidone carboxylates; polyethylene glycol; propylene glycol; 2-(2-ethoxyethoxy)ethanol; diethylene glycol monomethyl ether; N,N-dimethyldodecylamine-N-oxide and combinations of the foregoing. Alkylaryl ethers of polyethylene oxide, polyethylene oxide monomethyl ethers, and polyethylene oxide dimethyl ethers are also suitable, as are solubilizers such as glycerol and N-methyl pyrrolidone. The terpenes are another useful class of softeners, including pinene, d-limonene, carene, terpineol, terpinen-4-ol, carveol, carvone, pulegone, piperitone, menthone, menthol, neomenthol, thymol, camphor, bomeol, citral, ionone, and cineole, alone or in any combination. Of the terpenes, terpineol, particularly α-terpineol, is preferred.

Preferred softeners include glyceryl monolaurate, terpineol, lauryl alcohol, tetraglycol, tetraethylene glycol, propylene glycol, isopropyl myristate, ethyl oleate, methyl laurate, and 2-(2-ethoxyethoxy)ethanol.

While many of the softeners enumerated above are known to affect skin penetration rate, certain softeners affect aspects of performance other than and in addition to skin penetration rate. For example, they are useful in softening or increasing the compliance value and/or lowering the glass transition temperature of polymers, such that the resulting composition is more suitable for use as a pressure sensitive adhesive.

In a composition of the invention the softener(s) is dispersed, preferably substantially uniformly, and more preferably dissolved in the composition. Where the softener is a penetration enhancer, it is present in an amount that enhances drug permeation through the skin compared to a like composition not containing the penetration enhancer(s) when this phenomenon is measured using a standard skin penetration model, such as in U.S. Pat. No. 5,585,111 (Peterson), the disclosure of which is herein incorporated by reference. The total amount of softener will generally be about 5 to about 40% by weight based on the total weight of the composition.

In another aspect of the present invention, the base layer is prepared in one step. In another step, the liquid composition comprising at least one medicinal ingredient intended to have a medicinal or therapeutic effect is non-contact printed onto at least a portion of a flexible film. Subsequently, in a third step, the liquid composition is laminated to the base layer. This technique, where the liquid composition is non-contact printed onto a different layer than the base layer and subsequently laminated to the base layer will be referred to hereafter as the “indirect printing” technique.

Preparation of the base layer and the non-contact printing may be done by the methods described above used for direct printing.

As in direct printing, the viscosity of the liquid composition should be suitable for non-contact printing, and more preferably for ink jet printing as is conventionally known in the art. In addition, the viscosity and chemical composition of the liquid composition should be such as to allow it to remain substantially in place on the flexible film for a long enough time to allow for the subsequent lamination step. The length of time that the liquid remains substantially in place is dependent on the type of coating liquid, the type of film, and the amount of liquid. This time is preferably more than 1 second and more preferably more than 30 seconds. The flexible film may be any of the previously mentioned films suitable for use as the substrate for the base polymer.

In the third step, the liquid composition is laminated to the base layer, and at least a portion of the liquid is allowed to diffuse into the base layer until the prepared medical PSA article is sufficiently dry to allow wind-up or storage. The length of time that this takes is dependent on the type of base layer, the type of coating liquid, and the relative amounts of base layer and coating liquid. This time is preferably less than about 5 to about 10 minutes and more preferably less than about one minute. To facilitate and accelerate diffusion of the liquid composition, it may be advantageous to heat the base-layer just before, during, and/or after lamination with the liquid composition.

In yet another aspect of the present invention the base layer comprises both a polymer selected from the group consisting of acrylates, natural rubbers, synthetic rubbers such as polyisobutylenes, polyisoprenes, styrenic block copolymers, polyvinylethers, and silicone polymers and at least one medicinal ingredient intended to have a medicinal or therapeutic effect. The liquid composition optionally comprises a medicinal ingredient, and may consist solely of one or more plasticizers, liquid tackifiers, solvents, and combinations thereof. The liquid composition is non-contact printed onto the base layer and allowed to diffuse into at least a portion of the base layer using the direct printing technique.

Types of polymers, methods of preparation of the base layer, and non-contact printing methods are as described above. The medicinal ingredient may be mixed with the polymer of the base layer by any conventional method known to those skilled in the art. Most typically the medicinal ingredient may be mixed into a solution of the base polymer. This mixture is subsequently coated onto a substrate using the methods described above. In a preferred embodiment the base layer comprising the medicinal ingredient is non-tacky and the article becomes tacky when the liquid composition diffuses into the base layer.

In still another aspect of the present invention the base layer comprises both a polymer selected from the group consisting of acrylates, natural rubbers, synthetic rubbers such as polyisobutylenes, polyisoprenes, styrenic block copolymers, polyvinylethers, and silicone polymers and at least one medicinal ingredient intended to have a medicinal or therapeutic effect. The liquid composition optionally comprises a medicinal ingredient, and may consist solely of one or more plasticizers, liquid tackifiers, solvents, and combinations thereof. The liquid composition is coated onto a flexible film and laminated to the base layer to provide a medical pressure sensitive adhesive article using the indirect printing technique.

Types of polymers, methods of preparation of the base layer, and non-contact printing methods are as described above. In a preferred embodiment the base layer comprising the medicinal ingredient is non-tacky and the article becomes tacky when the liquid composition diffuses into the base layer.

### EXAMPLES

### Content Test Method A

The terpineol, propylene glycol, and testosterone content results given in the examples below were obtained using the following test method. The liner was removed from the patches and the patches were placed in a 236 mL jar. The backing and coating were extracted using 100 mL per 30 cm² patch area of a solution consisting of 67.5:32.5 by volume ethyl acetate (EtOAc):methanol (MeOH) containing 15.0 mg of decanol as an internal standard. The samples were shaken for 2 hours or until dissolved. An aliquot of this solution was then placed in an autosampler vial for analysis. Analysis of the samples was performed by gas chromatography with flame ionization detection (GC-FID) using a J&W DB-1 capillary column (30 m×0.53 mm i.d., 1.5 μm film) with helium carrier gas.

### Content Test Method B

The ethyl oleate content data given in the examples below was obtained using the following test method. The liner was removed from the patches and the patches were placed in a 236 mL jar. The backing and coating were extracted using 100 mL per 25 Cm² patch area of a solution consisting of 90.0:10.0 by volume ethyl acetate (EtOAc):methanol (MeOH) containing 31.3 mg of methyl stearate as an internal standard. The samples were sonicated for 1 hour or until dissolved. An aliquot of this solution was then placed in an autosampler vial for analysis. Analysis of the samples was performed by gas chromatography with flame ionization detection (GC-FID) using a Restek Rtx-200 capillary column (30 m×0.32 mm i.d., 1.0 μm film) with helium carrier gas.

### Probe Tack Test Method

The tack data given in the examples below was obtained using a Digital Polyken Probe Tack Tester, Model 80-02-01 (Testing Machines, Inc., Amityville, N.Y.). The machine settings were as follows: speed: 0.5 cm/second, dwell: 2 seconds; mode: peak; annulus: 20 g. A stainless steel probe was used. The result of the test is the force required to break the bond between the probe and the surface of the test sample. The force is measured in “grams of tack”. Reported results represent the average of the probe tack measured for 3 samples.

### Inherent Viscosity

The inherent viscosity values which are reported below were measured by conventional means using a Cannon-Fenske #50 viscometer in a water bath controlled at 27° C. to measure the flow time of 10 millimeters of the polymer solution (0.15 g of polymer per deciliter of ethyl acetate). The test procedure and apparatus are described in detail in Textbook of Polymer Science, F. W. Billmeyer, Wiley Interscience, Second Edition (1971), pages 84 and 85.

### Example 1

A solution was prepared by combining isooctyl acrylate, acrylamide, and vinyl acetate in a 71:9:20 (w:w) ratio in a reaction vessel. The solution was diluted to approximately 35% solids with an 80:20 (w:w) mixture of ethyl acetate and methanol. The solution was heated to 53° C. and 2,2′-azobis(2-methylbutyronitrile) was added. The reactor was purged thoroughly with nitrogen. Additional charges of 2,2′-azobis(2-methylbutyronitrile) dissolved in an 80:20 (w:w) mixture of ethyl acetate and methanol were added after 4 hours of reaction time and after 20 hours of reaction time. An additional 80:20 (w:w) mixture of ethyl acetate and methanol was added to reduce viscosity and aid mixing. The reaction was terminated when the percent residual isooctyl acrylate was 0.4% or less. The resulting base polymer solution was diluted with 80:20 (w:w) mixture of ethyl acetate and methanol to 15 to 17% solids. The diluted base polymer solution was knife coated onto a silicone coated release liner using a wet coating gap of 127 μm. The coated liner was oven dried at 70.0° C. for 15 minutes to prepare the base polymer layer. Polyken probe tack testing of the base polymer layer gave a tack value of 0 g and it did not adhere to skin.

The liquid composition was prepared by mixing propylene glycol with terpineol in a ratio of 20.8% to 79.2% by weight.

Ink jet printing was done with a Trident MicroCoder™ (Trident, Inc. of Brookfield, Conn.) printhead using a three orifice per channel chamber plate. The drive system included: an encoder for providing incremental pulses to the print head driver; a divider circuit for reducing the number of pulses to the encoder; digital circuitry for compression and decompression of the software image; temperature controls and heaters for the ink jet head; motion control hardware including a drum and a linear actuator for the ink jet head; and motion control software.

The propylene glycol/terpineol mixture was heated to approximately 50° C. in the ink jet head. Circular images were printed onto the base layer in sizes of either 20 cm² area or 40 cm² area. The printed samples were laminated to a silicone coated release liner.

Steel-rule dies were used to punch circular samples from the coated web. Small punches (5 cm²) were taken from the center of the printed area and larger punches were taken just inside and outside of the printed area. For the 20 cm² printed areas, punches of 5 cm², 19 cm², and 21 cm² were taken. For the 40 cm² printed areas, punches of 5 cm², 40 cm², and 46 cm² were taken.

Polyken probe tack testing of the printed samples gave a tack value of 430 g and the sample adheres to skin. The samples were analyzed for terpineol and propylene glycol content using Content Test Method A. The results are given in Table 1 below.

### Example 2

A solution was prepared by combining isooctyl acrylate (150.0 g), acrylamide (10.0 g), vinyl acetate (40.0 g), 2,2′-azobis(2,4-dimethylpentanenitrile) (0.200 g), ethyl acetate (323.6 g) and methanol (32.0 g) in a 1 quart (0.95 L) amber glass bottle. The bottle was purged for 2 minutes with nitrogen at a flow rate of 1 L per minute. The bottle was sealed and placed in a rotating water bath at 45° C. for 24 hours. At 24 hours the bottle was removed from the rotating water bath and unsealed. The resulting copolymer was diluted with ethyl acetate (62.5 g) and methanol (6.94 g) to 26.3% solids. The inherent viscosity was 1.25 dL/g. A base polymer coating was prepared by knife coating this solution onto a silicone release liner using a wet coating gap of 152 μm. The coated liner was oven dried at 70.0° C. for 15 minutes to prepare the base polymer layer. Polyken probe tack testing of the base polymer layer gave a test value of 654 g.

The liquid composition was ethyl oleate.

Ink jet printing was done using the same method as described in Example 1 with the exception that the ethyl oleate was dispensed from the ink jet head at room temperature (approximately 23° C.). Polyken probe tack testing of the printed samples gave a tack value of 317 g. Samples were punched as described in Example 1 and analyzed for ethyl oleate content using Content Test Method B. The results are given in table 2 below.

### Example 3

A solution was prepared by combining isooctyl acrylate (83.75 g), acrylamide (16.25 g), vinyl acetate (25.0 g), 2,2′-azobis(2-methylbutyronitrile) (0.1875 g), ethyl acetate (110.65 g) and methanol (36.69 g) in a 1 quart (0.95 L) amber glass bottle. The bottle was purged for 2 minutes with nitrogen at a flow rate of 1 L per minute. The bottle was sealed and placed in a rotating water bath at 53° C. for 24 hours. At 24 hours the bottle was removed from the rotating water bath and unsealed. The resulting copolymer was diluted with ethyl acetate (171.195 g) and methanol (57.065 g) to 19.8% solids. The inherent viscosity was 1.23 dL/g. Dried copolymer was prepared by knife coating this solution onto a release liner. The copolymer coated release liner was oven dried at an elevated temperature to remove solvent and reduce the level of residual monomers. The dried copolymer was then stripped off the release liner and stored in a container.

Dried copolymer (9.995 g), ethyl acetate (9.081 g), and methanol (6.068 g) were combined and mixed until the solution was clear and homogenous to prepare a base polymer coating solution. The base polymer coating solution was knife coated at a wet thickness of 254 μm onto a release liner (Daubert 164P silicone coated release liner). The coated liner was oven dried for 4 minutes at 43° C., for 2 minutes at 85° C., and for 2 minutes at 107° C. The dry coated weight of the resulting base polymer layer was 5.0 mg/cm². Polyken probe tack testing of the base polymer layer gave a test value of 0 g and it did not adhere to skin.

The liquid composition was prepared by combining terpineol (50.501 g), propylene glycol (13.764 g), and testosterone (10.723 g). This combination was mixed until it became clear and homogenous.

Ink jet printing was done using the same method as described in Example 1 with the exception that the printed area was controlled to be a square with sides of 4.3 cm length. Samples were cut along the edges of the printed area and analyzed for terpineol, propylene glycol, and testosterone content using Content Test Method A. Polyken probe tack testing of the printed samples gave a tack value of 575 g and the sample sticks to skin.

### Example 4

The dried copolymer of Example 3 (4.615 g) was combined with testosterone (0.384 g), ethyl acetate (7.043 g), and methanol (4.700 g) and mixed until the solution was clear and homogenous to prepare a base polymer coating solution. The base polymer coating solution was knife coated at a wet thickness of 279 μm onto a release liner (Daubert 164P silicone coated release liner). The coated liner was oven dried for 4 minutes at 43° C., for 2 minutes at 85° C., and for 2 minutes at 107° C. The dry coated weight of the resulting base polymer layer was 5.1 mg/cm². Polyken probe tack testing of the base polymer layer gave a test value of 0 g and it did not adhere to skin.

The liquid composition was prepared by combining terpineol (59.044 g) and propylene glycol (16.349 g). This solution was mixed until it became clear and homogenous.

Ink jet printing was done using the same method as described in Example 3. Samples were cut along the edges of the printed area and analyzed for terpineol, propylene glycol, and testosterone content using Content Test Method A. Polyken probe tack testing of the printed samples gave a tack value of 667 g.

The present invention has been described with reference to several embodiments thereof. The foregoing detailed description and examples have been provided for clarity of understanding only, and no unnecessary limitations are to be understood therefrom. It will be apparent to those skilled in the art that many changes can be made to the described embodiments without departing from the spirit and scope of the invention. Thus, the scope of the invention should not be limited to the exact details of the compositions and structures described herein, but rather by the language of the claims that follow.

What is claimed is:

1. A method of preparing a medical pressure sensitive adhesive article comprising:
(a) providing a base layer on at least a portion of a substrate, wherein the base layer comprises a polymer,
(b) non-contact printing a liquid composition comprising at least one medicinal ingredient intended to have a medicinal or therapeutic effect onto at least a portion of the base layer, and
(c) allowing at least a portion of the liquid composition to diffuse into the base layer to provide a medical pressure sensitive adhesive article.

2. The method of claim 1 wherein the polymer of the base layer is an acrylate copolymer.

3. The method of claim 1 wherein the base layer is a pressure sensitive adhesive.

4. The method of claim 1 wherein the substrate is a flexible film and the article is a sheet.

5. The method of claim 4 wherein the flexible film is provided as a continuous web, wherein the liquid composition is printed in island patterns onto the web, and further comprising the step of applying a release liner to the surface of the base layer opposed to the substrate to provide a pattern printed rollstock.

6. The method of claim 4 wherein the flexible film is provided as a continuous web, wherein the liquid composition is printed in island patterns onto the web, and further comprising the step of converting the printed island patterns into discrete pressure sensitive tape patches.

7. The method of claim 6 wherein the pressure sensitive tape patches are transdermal drug delivery devices.

8. The method of claim 1 wherein the base layer is non-tacky.

9. The method of claim 1 wherein the medicinal ingredient intended to have a medicinal or therapeutic effect is a drug.

10. The method of claim 9 wherein the polymer of the base layer is an acrylate copolymer.

11. The method of claim 10 wherein the substrate is a flexible film and the article is a sheet.

12. The method of claim 11 wherein the flexible film is provided as a continuous web, wherein the liquid composition is printed in island patterns onto the web, and further comprising the step of converting the printed island patterns into discrete pressure sensitive tape patches.

13. The method of claim 1 wherein the medicinal ingredient intended to have a medicinal or therapeutic effect is selected from the group consisting of antimicrobials, antifungals, anti-irritants, and cosmetic agents.

14. A method of transdermal delivery of a drug comprising:
(a) providing a base layer on at least a portion of a substrate, wherein the base layer comprises a polymer,
(b) non-contact printing a liquid composition comprising a drug onto at least a portion of the base layer and allowing the liquid composition to diffuse into the base layer to provide a transdermal drug delivery device, and
(c) applying the transdermal drug delivery device to an external part of the human body for a period of time sufficient to achieve the desired therapeutic result.

15. A method of transdermal delivery of a drug comprising:
(a) coating a base layer on at least a portion of a substrate, wherein the base layer comprises a polymer,
(b) non-contact printing a liquid composition comprising a drug onto at least a portion of the base layer and allowing the liquid composition to diffuse into the base layer to provide a transdermal drug delivery device, and
(c) applying the transdermal drug delivery device to an external part of the human body for a period of time sufficient to achieve the desired therapeutic result.

16. A method of preparing a medical pressure sensitive adhesive article comprising:
(a) providing a base layer on at least a portion of a substrate, wherein the base layer comprises a polymer,
(b) non-contact printing a liquid composition comprising a medicinal ingredient intended to have a medicinal or therapeutic effect onto at least a portion of a flexible film,
(c) laminating the base layer to the printed liquid composition, and
(d) allowing at least a portion of the liquid composition to diffuse into the base layer to provide a medical pressure sensitive adhesive article.

17. The method of claim 16 wherein the polymer of the base layer is an acrylate polymer.

18. The method of claim 16 wherein the medicinal ingredient intended to have a medicinal or therapeutic effect is selected from the group consisting of drugs, antimicrobials, antifungals, anti-irritants, and cosmetic agents.

19. The method of claim 16 wherein the medicinal ingredient intended to have a medicinal or therapeutic effect is a drug.

20. The method of claim 16 wherein the base layer is a pressure sensitive adhesive.

21. The method of claim 16 wherein the base layer is non-tacky.

22. The method of claim 16 wherein the substrate is a flexible film and the article is a sheet.

23. The method of claim 22 wherein the flexible film is provided as a continuous web, wherein the liquid composition is printed in island patterns onto the web, and further comprising the step of converting the printed island patterns into discrete pressure sensitive tape patches.

24. The method of claim 23 wherein the pressure sensitive tape patches are transdermal drug delivery devices.

25. A method of preparing a medical pressure sensitive adhesive article comprising:
(a) providing a base layer on at least a portion of a substrate, wherein the base layer comprises a polymer and at least one medicinal ingredient intended to have a medicinal or therapeutic effect,
(b) non-contact printing a liquid composition onto at least a portion of the base layer, and
(c) allowing at least a portion of the liquid composition to diffuse into the base layer to provide a medical pressure sensitive adhesive article.

26. The method of claim 25 wherein the polymer of the base layer is an acrylate copolymer.

27. The method of claim 25 wherein the substrate is a flexible film and the article is a sheet.

28. The method of claim 27 wherein the flexible film is provided as a continuous web, wherein the liquid composition is printed in island patterns onto the web, and further comprising the step of converting the printed island patterns into discrete pressure sensitive tape patches.

29. The method of claim 25 wherein the base layer is non-tacky.

30. The method of claim 25 wherein the medicinal ingredient intended to have a medicinal or therapeutic effect is a drug.

31. The method of claim 25 wherein the medicinal ingredient intended to have a medicinal or therapeutic effect is selected from the group consisting of antimicrobials, antifungals, anti-irritants, and cosmetic agents.

32. A method of preparing a medical pressure sensitive adhesive article comprising:
(a) providing a base layer on at least a portion of a substrate, wherein the base layer comprises a polymer and at least one medicinal ingredient intended to have a medicinal or therapeutic effect,
(b) non-contact printing a liquid composition onto at least a portion of a flexible film,
(c) laminating the base layer to the printed liquid composition, and
(d) allowing at least a portion of the liquid composition to diffuse into the base layer to provide a medical pressure sensitive adhesive article.

33. The method of claim 32 wherein the polymer of the base layer is an acrylate copolymer.

34. The method of claim 32 wherein the substrate is a flexible film and the article is a sheet.

35. The method of claim 34 wherein the flexible film is provided as a continuous web, wherein the liquid composition is printed in island patterns onto the web, and further comprising the step of converting the printed island patterns into discrete pressure sensitive tape patches.

36. The method of claim 32 wherein the base layer is non-tacky.

37. The method of claim 32 wherein the medicinal ingredient intended to have a medicinal or therapeutic effect is a drug.

38. The method of claim 32 wherein the medicinal ingredient intended to have a medicinal or therapeutic effect is selected from the group consisting of antimicrobials, antifungals, anti-irritants, and cosmetic agents.
